(19) United States
(12) Patent Application Publication
(10) Pub. No.: US 2006/0272038 A1
(43) Pub. Date: Nov. 30, 2006

(54) Transgenic Alzheimer's mouse model vectors and uses thereof

(76) Inventors: Michael De Vivo, New York, NY (US); Joerg August Konrad Heyer, Cambridge, MA (US); Mei Yee Kwan, Tuxedo Park, NY (US); Daguang Wang, New York, NY (US); Qiurong Xiao, West Roxbury, MA (US)

Correspondence Address: DOCKET ADMINISTRATOR;LOWENSTEIN SANDLER PC, 65 LIVINGSTON AVENUE, ROSELAND, NJ, 07068, US

(21) Appl. No.: 11/441,744

(22) Filed: May 26, 2006

Related U.S. Application Data
(60) Provisional application No. 60/685,649, filed on May 27, 2005.

(51) Int. Cl. A01K 67/027; C07H 21/04; C12P 21/06; C12N 9/64; C12N 15/00; C12N 5/06; C12N 5/08

(52) U.S. Cl. 800/12; 800/18; 435/69.1; 435/368; 435/354; 435/226; 435/320.1; 536/23.2

(57) ABSTRACT

The present invention provides for a recombinant nucleic acid molecule comprising a humanized mouse β-amyloid precursor protein (“APP”) gene comprising K670N, M671L and V717F mutations and uses thereof. The present invention further provides for a recombinant nucleic acid molecule comprising a region of a calcium-calmodulin dependent kinase IIα (“CaMKIIα”) promoter operatively linked to a β-amyloid precursor protein (“APP”) gene comprising at least one mutation and uses thereof.

### CROSS-REFERENCE TO RELATED APPLICATION

This application claims priority benefit of U.S. Provisional Application Ser. No. 60/685,649, filed May 27, 2005, which is incorporated herein by reference in its entirety.

### FIELD OF THE INVENTION

The present invention provides for a recombinant nucleic acid molecule comprising a humanized mouse β-amyloid precursor protein (“APP”) gene comprising K670N, M671L and V717F mutations and uses thereof. The present invention further provides for a recombinant nucleic acid molecule comprising a region of a calcium-calmodulin dependent kinase IIα (“CaMKIIα”) promoter operatively linked to a β-amyloid precursor protein (“APP”) gene comprising at least one mutation and uses thereof.

### BACKGROUND OF THE INVENTION

Throughout this application, various publications are referenced by author and date. The disclosures of these publications in their entireties are hereby incorporated by reference into this application in order to more fully describe the state of the art as known to those skilled therein as of the date of the invention described and claimed herein.

Alzheimer's Disease (AD) is a human disease for which there is currently no effective treatment. AD is characterized by progressive impairments in memory, behavior, language, and visuo-spatial skills, typically progressing in severity over a 6 to 20-year period, ending in death.

The neocortex, amygdala and hippocampus of the brain are the primary sites of neuropathology in AD. The typical neuropathology of AD comprises extracellular neuritic plaques, intracellular neurofibrillary tangles, neuronal cell loss, gliosis and cerebral vessel amyloid deposition. The neuritic plaques consist of cores of amyloid protein fibrils surrounded by a rim of dystrophic neurites; the plaques have been suggested as the primary site of damage to the cortex. The major protein component of the amyloid protein of the plaque is known as the Aβ peptide, a 4 kD peptide comprising between 39 and 43 amino acids. The Aβ peptide that predominates in plaques has 40 or 42 amino acids.

The Aβ peptide is proteolytically derived from an integral membrane protein known as the β-amyloid precursor protein (“APP”). There are several APP isoforms (having 695, 751 or 770 amino acids), which are encoded by mRNA species resulting from alternative splicing of a common precursor RNA. Standard numbering for the APP isoforms is in accordance with the isoform having 770 amino acids, and this convention is used even when referring to codon positions of the shorter isoforms. The APP gene is encoded by a single copy gene found on human chromosome 21 (Estus et al., Science 255:726-728 (1992)). The APP gene product (“APP”) is alternatively processed via two cellular pathways. Processing in the “amyloidogenic” pathway yields APP fragments bearing the Aβ peptide or the Aβ peptide itself. Alternatively, in the “nonamyloidogenic” pathway, APP is cleaved within the Aβ sequence. This results in destruction of the Aβ peptide and secretion of the large N-terminal ectodomain of APP. The Aβ peptide is produced and secreted by a wide variety of cell types in various animal species. It has been found in body fluids, including serum and cerebral spinal fluid.

Complementary DNAs encoding human APP, have been cloned and sequenced. See, e.g., Kang et al., Nature 325: 733-736 (1987); Goldgaber et al., Science 235:877-880 (1987); Tanzi et al., Nature 331:528-530 (1988); and Robakis et al., Proc. Natl. Acad. Sci. USA 84:4190-4194 (1987). The cDNA for a mouse homolog of human APP has also been cloned and sequenced. Human and murine APP amino acid sequences have a high degree of homology (96.8%), indicating that the protein is conserved across mammalian species (Yamada et al., Biochem. Biophys. Res. Commun. 149: 665-671 (1987)). The mouse Aβ and human Aβ sequences differ at positions 5, 10 and 13 (i.e., positions 676, 681 and 684 of the complete APP770 sequence). The amino acid changes, from mouse to human Aβ, are: Gly to Arg (Aβ5, APP 676); Phe to Tyr (Aβ10, APP 681); and Arg to His (Aβ 13, APP 684). A “humanized mouse APP gene” is a mouse APP gene including the following mutations: G676R, F681Y and R684H.

A form of Alzheimer's disease known as “Swedish Familial Alzheimer's Disease” has been associated with two mutations known as the “Swedish FAD mutations.” The Swedish FAD mutations are transversions (G to T and A to C) in codons 670 and 671 (APP 770 transcript), which are in exon 16 of the APP gene (Mullan, Nature Genetics 1:345-347 (1992)). The Swedish FAD mutations change lysine to asparagine (K670N) and methionine to leucine (M671L) at positions 670 and 671, respectively, in the amyloid precursor protein. These amino acid changes are immediately adjacent to the amino terminus of the Aβ peptide.

The Swedish FAD mutations may act by altering the proteolytic processing of APP so that increased amounts of Aβ are released (Cai et al., Science 259:514-516 (1993)). In vitro studies have demonstrated that cells expressing APP with the Swedish FAD mutation produce 3 to 7-fold more Aβ than cells expressing APP without the mutation.

Furthermore, it was shown that a familial form of Alzheimer's disease in an Indiana kindred has been associated with one mutation known as the “Indiana FAD mutation.” The Indiana FAD mutation is also a transversion (G to T) in codon 717, which results in a change of valine to phenylalanine (V717F) at position 717 (APP770 transcript) in the amyloid precursor protein (Zeldenrust, et al., J Med Genet. 30(6): 476-8 (1993)).

Other mutations in the APP gene have been associated with the Alzheimer's disease phenotype and are summarized in Table 1 (all in accordance with the APP 770 isoform):

TABLE 1

| Codon | Mutation* ** | Name | Phenotype |
|---|---|---|---|
| 670/671 | K -> N/M->L | Swedish | FAD, increased Aβ342 |
| 692 | A->G | Flemish | FAD, increased Aβ342, |
| | | | cerebral hemorrhage |
| 693 | E->G | | Late onset AD, not an |
| | | | inherited mutation |
| | E->Q | Dutch | Amyloidosis of the |
| | | | Dutch type |
| 713 | A->T | | AD, not an inherited |
| | | | mutation |
| 716 | I->V | | FAD |
| 717 | V->I | | FAD, increased long |
| | | | Aβ isoforms |
| | V->F | Indiana | FAD |
| | V->G | | FAD |

*indicating single amino acid substitutions which result from the gene mutations
** single letter amino acid designations

Genetically engineered nonhuman mammals may serve as models for at least some aspects of AD. The term “transgenic” has sometimes been used in a broad sense, to indicate any organism into which an exogenous piece of DNA has been incorporated. As used herein, however, the term “transgenic” is reserved for organisms (i.e., non-human mammals) comprising a piece of exogenous DNA that has been randomly inserted. A transgenic organism expresses the transgene in addition to all normally-expressed native genes (except the gene or genes in which the random insertion(s) may have taken place).

The genetic engineering of nonhuman mammals (or any other organism) may be carried out according to at least two fundamentally different approaches: (1) random insertion of an exogenous gene into a host organism, and (2) gene targeting. Transgenic non-human mammals resulting from the random insertion technique and comprising human APP DNA sequences, in addition to the native APP DNA sequences, are known. See, e.g., Quon et al., Nature 352: 239-241 (1991); Higgins et al., Annals NY Acad Sci. 695:224-227 (1994); Sandhu et al., J. Biol. Chem. 266:21331-21334 (1991); Kammesheid et al., Proc. Natl. Acad. Sci. USA 89:10857-10861 (1992); Lamb et al., Nature Genet. 5:22-30 (1993); Pearson et al., Proc. Natl. Acad. Sci. USA 90:10578-10582 (1993); McConlogue et al., McConlogue et al., Neurobiol. Aging 15, s12 (1994); Games et al., Nature 373:523-527 (1995); and U.S. Pat. No. 5,387,742.

Transgenic non-human mammals resulting from the gene targeting technique, wherein a selected native DNA sequence or gene (i.e., targeted gene) is partially or completely removed or replaced through a process known as homologous recombination are also known. One advantage of this technique is that if the targeted gene is a single-copy gene and the organism is homozygous at that locus, the gene-targeted organism can no longer express the targeted native gene, which can sometimes interfere and/or complicate transgenic studies. An attempt to produce, by gene targeting, mice homozygous for an APP null allele (and thus devoid of APP), has been published (Muller et al., Cell 79:755-765 (1994)). Also, a humanized APP gene-targeted transgenic mouse was produced expressing the Swedish FAD mutation (Reaume, et al., J. Bio. Chem. 271(38): 23380-2888 (1996)).

Transgenic mice expressing only one FAD mutation develop an AD phenotype very late in their lifespan, usually greater than 12 to 18 months. More recent studies have demonstrated that transgenic AD mouse models expressing transgenes with two mutations, for example, the Indiana and Swedish mutations, show an earlier onset of the AD phenotype at approximately less than six months of age (Chishti, et al., J. Biol. Chem. 276(24):21562-21570 (2001)).

It is desirable to construct a recombinant nucleic acid molecule to be used in developing an AD transgenic mouse model that results in early onset of the AD phenotype and the stable overexpression of the humanized APP gene.

### SUMMARY OF THE INVENTION

The present invention provides for a recombinant nucleic acid molecule comprising a humanized mouse β-amyloid precursor protein (“APP”) gene comprising K670N, M671L and V717F mutations and uses thereof. The present invention further provides for a recombinant nucleic acid molecule comprising a region of a calcium-calmodulin dependent kinase IIα (“CaMKIIα”) promoter operatively linked to a β-amyloid precursor protein (“APP”) gene comprising at least one mutation and uses thereof. Recombinant nucleic acid molecules of the invention may be advantageous in producing an early onset of AD phenyotype and stable overexpression of the humanized APP gene.

Other features and advantages of the invention will be apparent from the following description of the embodiments thereof, and from the claims.

### BRIEF DESCRIPTION OF THE FIGURES

FIG. 1 Comparison of the C-terminal region of the human, mouse and rat APP amino acid sequences. The Aβ42 peptide is underlined. Mutations are indicated by stars (see Table 1 for names).

FIG. 2 Comparison of the c-terminal regions of the human APP, mouse APP and the humanized mouse APP amino acid sequences encoded from the respective genes, including the Swedish and Indiana mutations. Mutations are indicated on the squares or circles with the circles representing the Swedish (SW) or Indiana (In) mutations and the squares representing the humanized amino acids. The numbering scheme is in accordance with the 695 APP isoform. The Swedish mutation K595N and M596L (695 isoform) and Indiana mutation V642F (695 isoform) are analogous to the K670N and M671L (770 isoform) and V717F (770 isoform), respectively. The mutations 601R, F606Y and R609H (695 isoform) are analogous to G676R, F6814Y, and R684H (770 isoform), respectively.

FIG. 3 Sequence of the hm APP (In−SW)-hs3′UTR gene.

FIG. 4 Sequence of the CaMKIIα promoter and hmAPP (In−Sw)-hs3′UTR transgene.

FIG. 5 The amino acid sequence encoded from the hmAPP(In−Sw) gene sequence.

### DETAILED DESCRIPTION

The present invention provides for a recombinant nucleic acid molecule comprising a humanized mouse β-amyloid precursor protein (“APP”) gene comprising K670N, M671L and V717F mutations and uses thereof. The present invention further provides for a recombinant nucleic acid molecule comprising a region of a calcium-calmodulin dependent kinase IIα (“CaMKIIα”) promoter operatively linked to a β-amyloid precursor protein (“APP”) gene comprising at least one mutation and uses thereof.

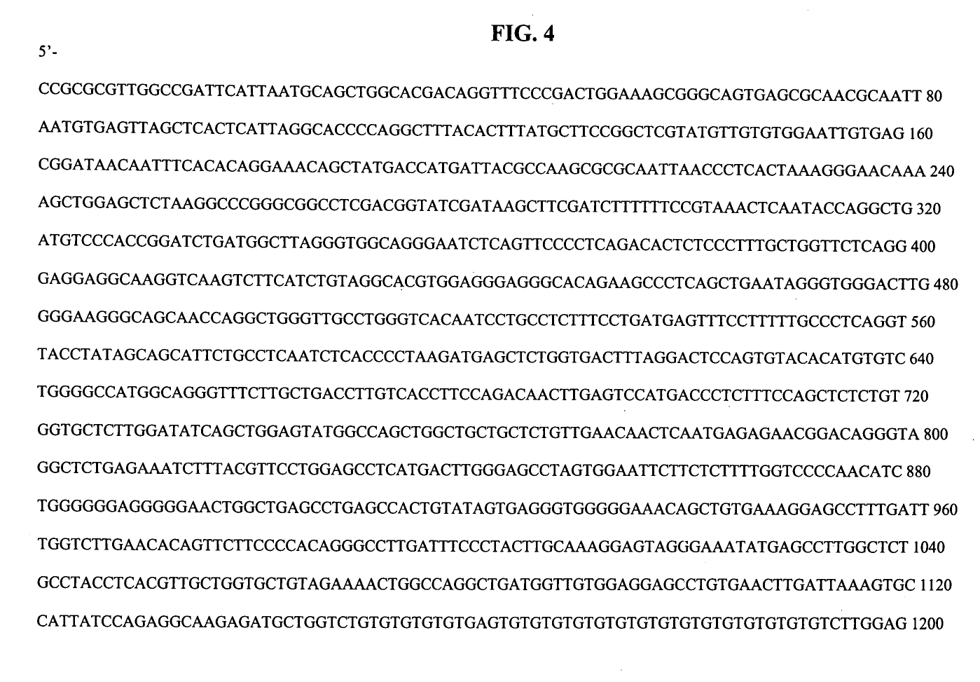

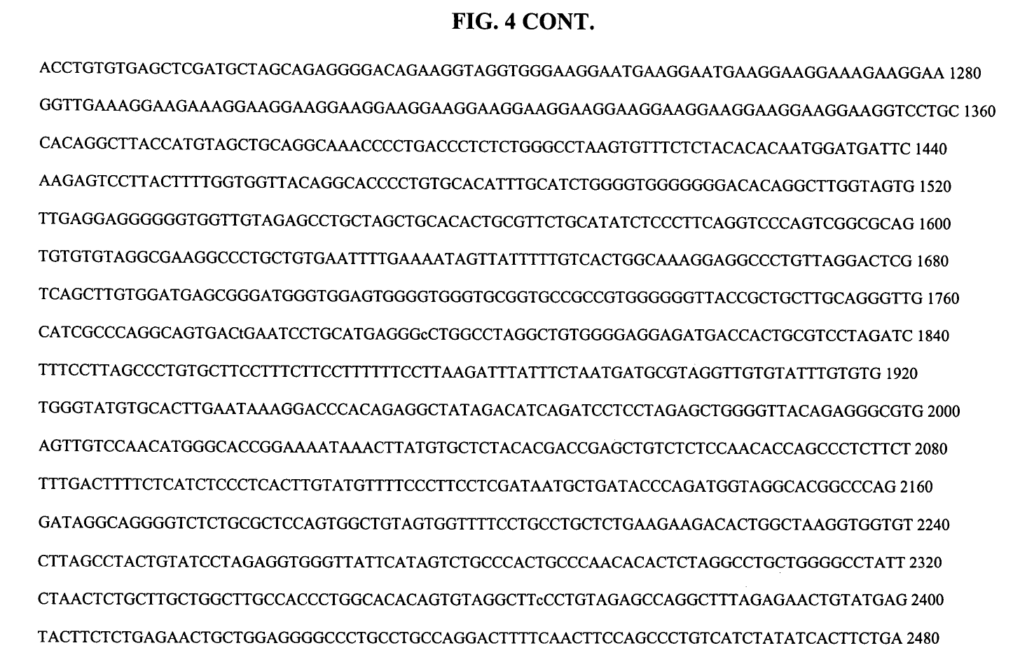

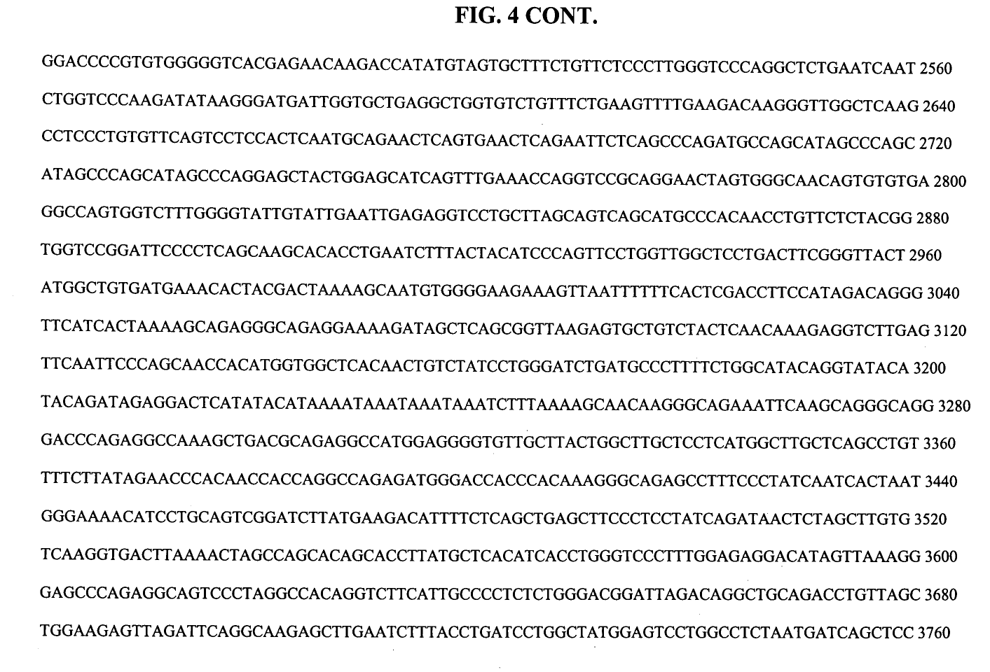

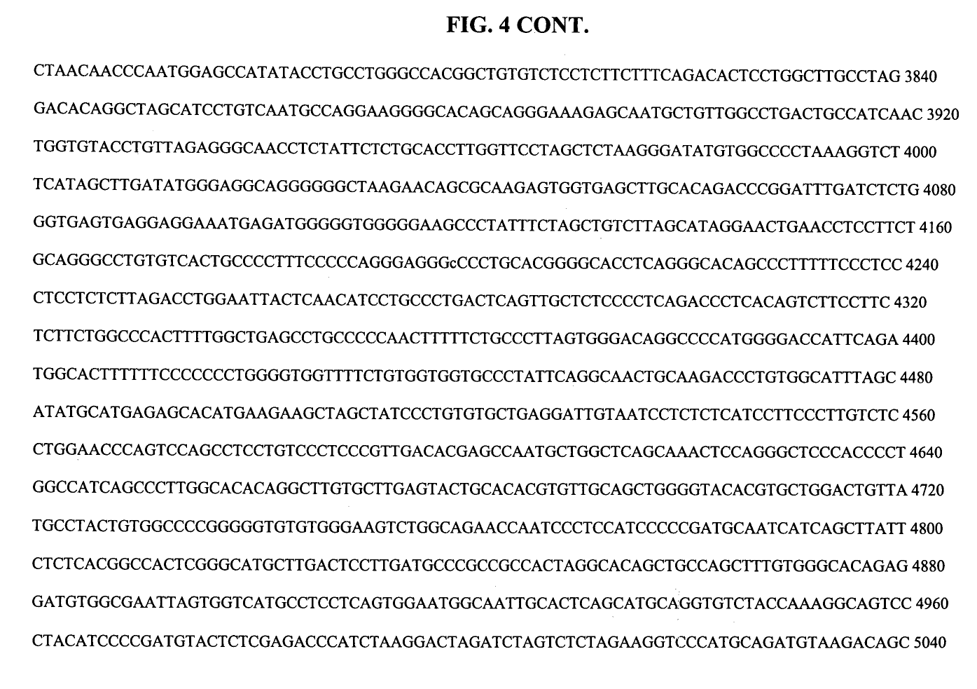

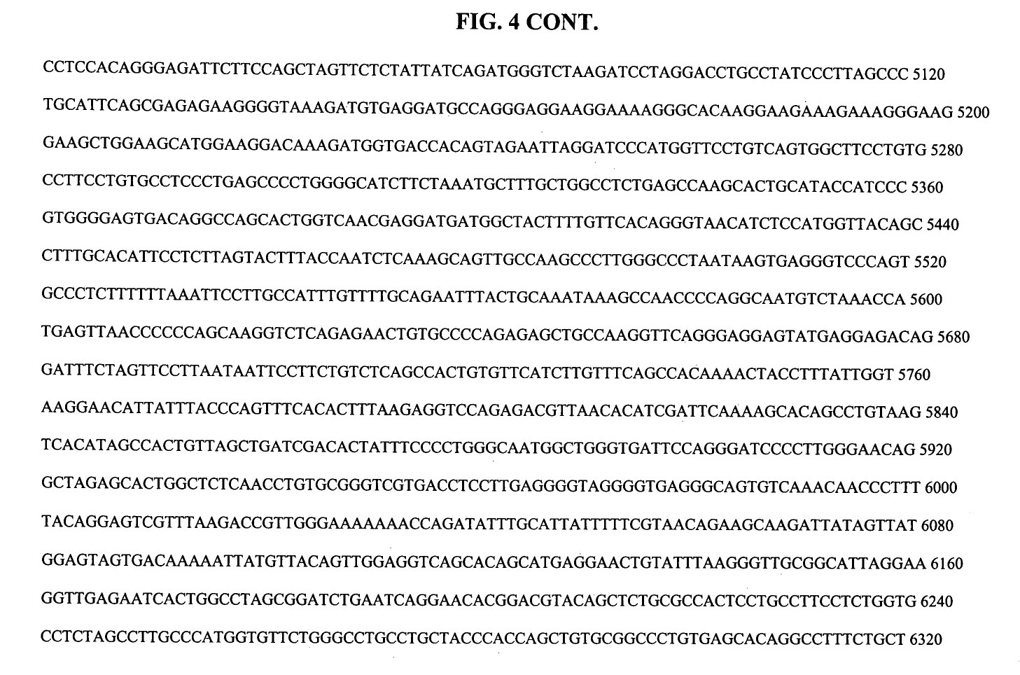

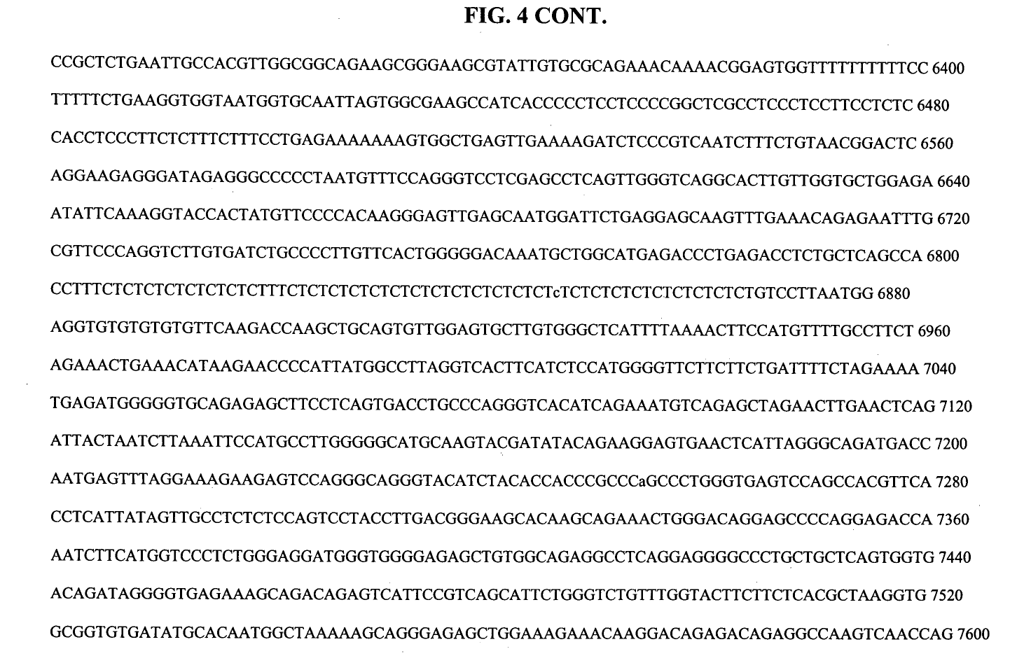

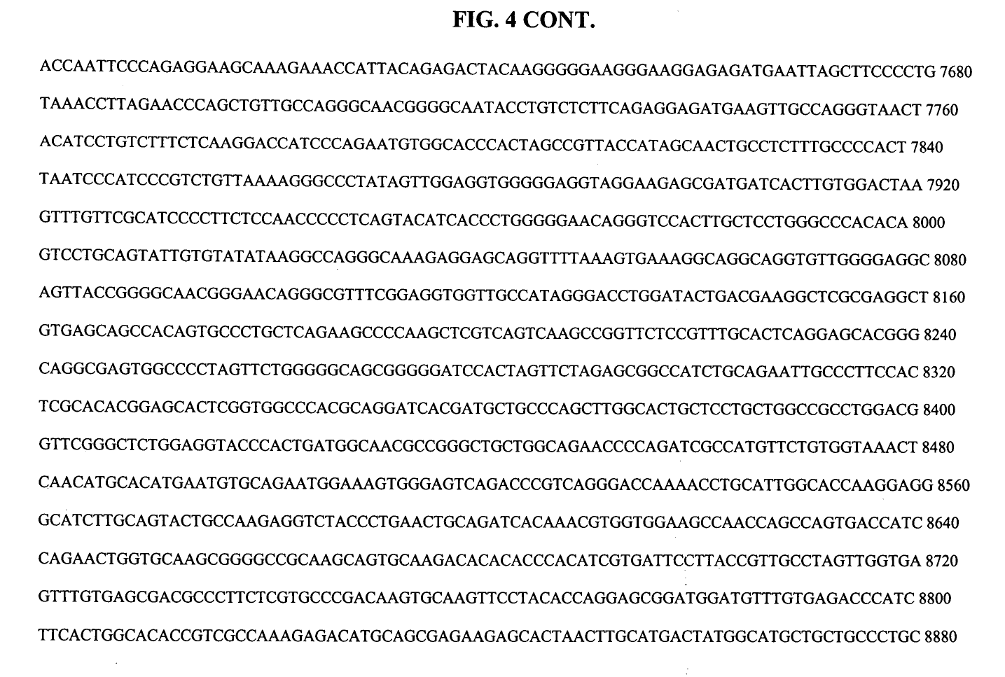

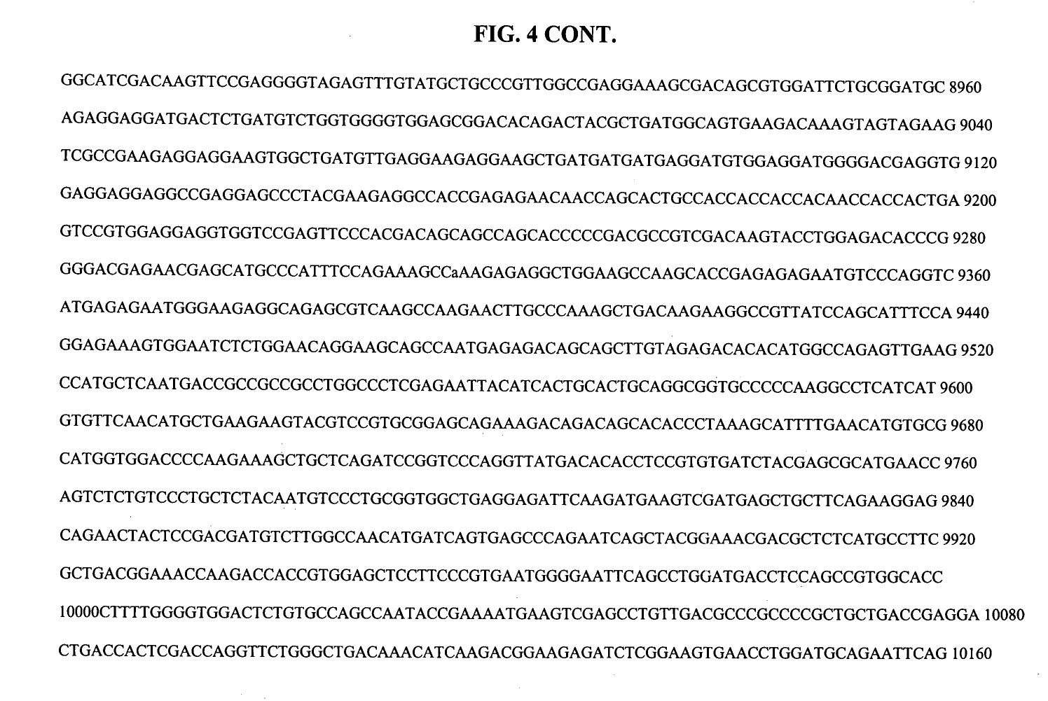

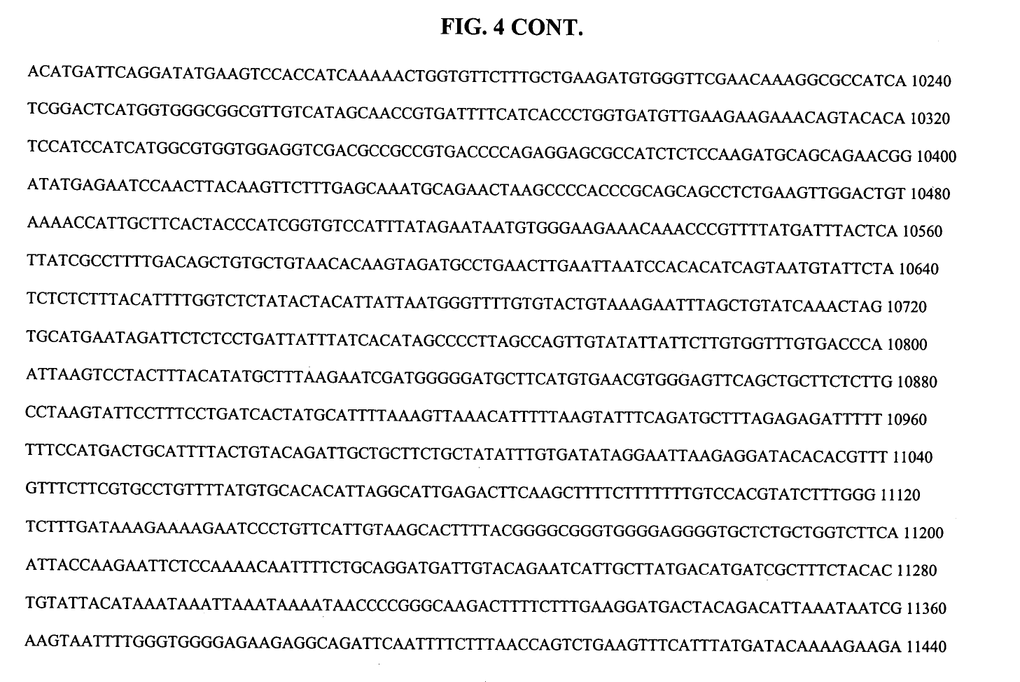

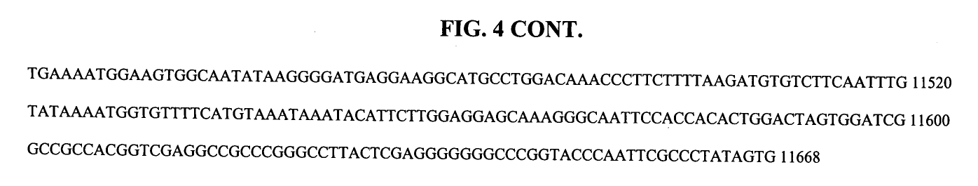

The CaMKIIα promoter, described in U.S. Pat. No. 6,509,190, which is incorporated herein by reference, specifically localizes expression of the gene of interest to the hippocampal region of the brain of a mammal. The use of the CaMKIIα promoter is advantageous because it provides brain-specific gene expression and may provide an increase in gene transcription and minimize side effects observed in current transgenic models. The nucleic acid sequence of the CaMKIIα promoter is set forth in FIG. 4 (nucleic acid number 1 to 8299).

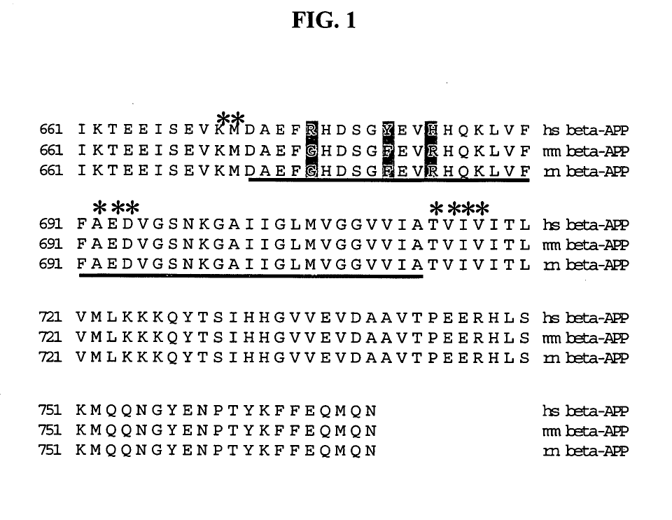

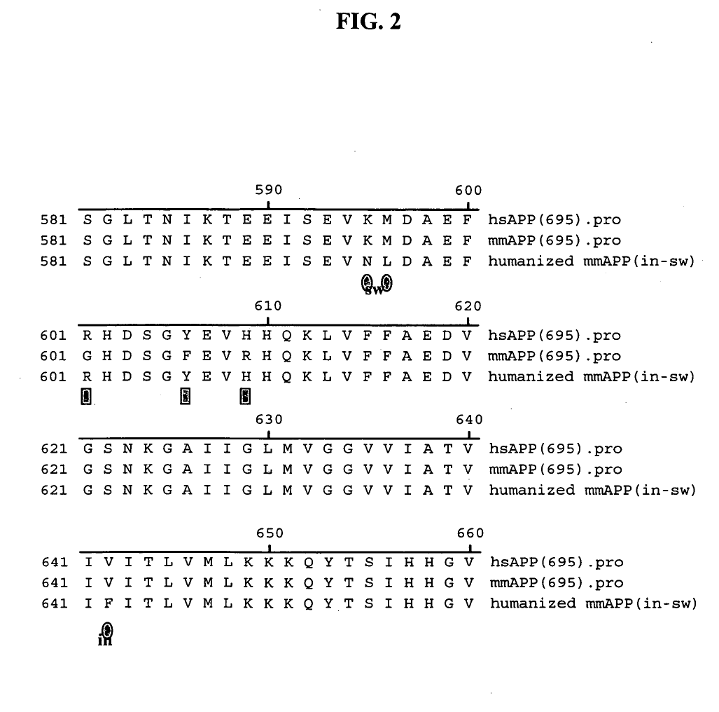

Most attempts to generate an AD mouse transgenic mouse model have utilized the human APP gene. As shown in FIG. 1, the rat and mouse amino acid sequences are 97% identical when compared to the human amino acid sequence. The differences are mostly found in the N-terminal region of the amino acid sequence. The C-terminus, where the Aβ42 peptide (underlined in FIG. 1) is generated, is identical among all three species with the exception of three amino acid changes at position 676, 681, and 684, respectively (indicated by shaded boxes in FIG. 1). It is therefore conceivable to utilize the mouse or rat protein without compromising the disease-generating ability of the protein, if the amino acids are changed back to the human sequence (i.e., humanized mouse APP gene). Use of humanized mouse APP gene allows researchers to differentiate the function of the transgene from the native mouse APP gene.

An embodiment of the present invention is a recombinant nucleic acid molecule comprising humanized mouse APP comprising a 670N, M671L and V717F mutations. The embodiment further comprises a CaMKIIα promoter operatively linked to the humanized mouse APP gene. A further embodiment of the present invention is the recombinant nucleic acid molecule further comprising a region of 3′ in translated region (“3′ UTR”). The 3′ UTR can be, but is not limited to, an APP 3′ UTR or a human APP 3′ UTR. It has been reported that the 3′ UTR can elevate the expression of the human APP gene by more than two-fold.

A further embodiment of the present invention is a recombinant nucleic acid molecule CaMKIIhmAPPS1, comprising CaMKIIα promoter operatively linked to a humanized mouse APP gene comprising K670N and M671L mutations (i.e., the Swedish mutations) and a V717F mutation (i.e., the Indiana mutation) and a region of the human APP 3′-UTR, corresponding to the nucleic acid sequence of ATCC Accession No. PTA-6646, which was deposited on Mar. 29, 2005 under provisions of the Budapest Treaty with the American Type Culture Collection (see details hereinbelow).

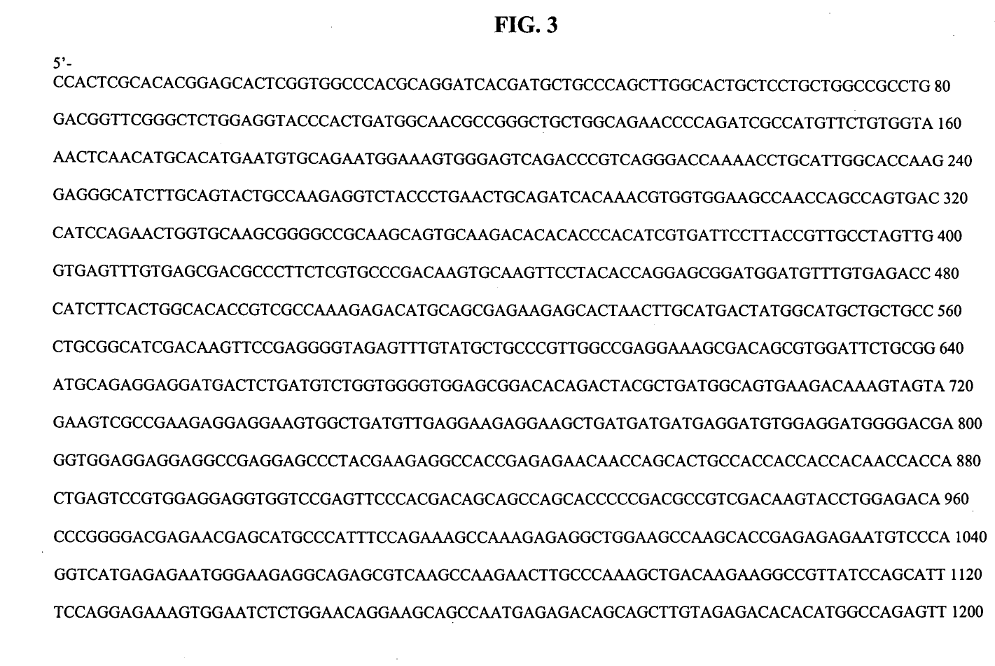

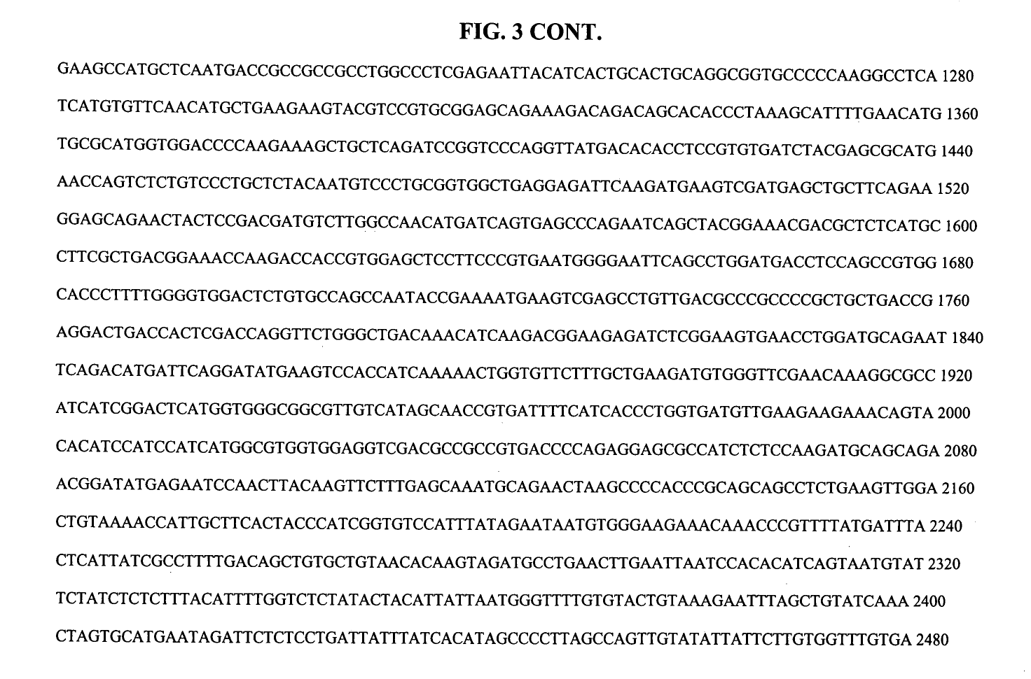

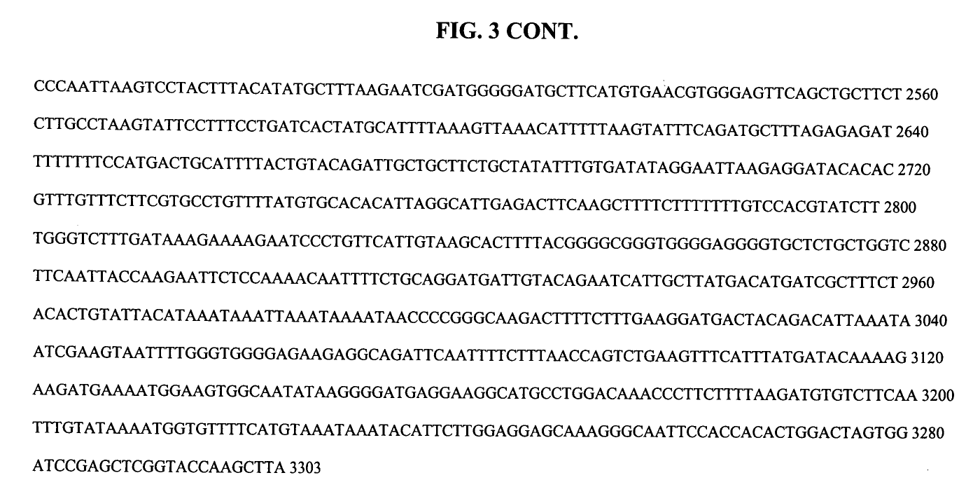

A further embodiment is a recombinant nucleic acid molecule that has a sequence which comprises the sequence in FIG. 3 and FIG. 4.

The present invention also provides for a recombinant nucleic acid molecule comprising a CaMKIIα promoter operatively linked to a APP gene comprising at least one mutation. In one embodiment, the mutation confuses K670N and M671L. In another embodiment, the mutation confuses K670N, M671L, and V717F. In a further embodiment, the APP gene is a humanized mouse APP gene. Another embodiment of the present invention provides for a recombinant nucleic acid molecule comprising a CaMKIIα promoter operatively linked to an APP gene comprising at least one mutation and a region of a 3′-UTR. The 3′ UTR can be, but is not limited to, an APP 3′ UTR or a human APP 3′ UTR.

The mutation in the APP gene can be any of the mutations listed in Table 1 or any combination thereof. It is readily appreciated by the skilled artisan that the representations of the different mutations represent single amino acid substitutions. For example, K670N refers to a native amino acid, lysine (“K”), substituted by an amino acid, asparagine (“N”), at position 670 of the APP amino acid sequence resulting from a mutation in the APP gene sequence.

For example, embodiments of the invention include, but are not limited to, a recombinant nucleic acid molecule comprising CaMKIIα promoter operatively linked to a APP gene a comprising A692G mutation, the “Flemish” mutation, and a V717F mutation, the Indiana mutation and a region of the human APP 3′-UTR. The present invention further provides for a recombinant nucleic acid molecule comprising CaMKIIα promoter operatively linked to an APP gene comprising K670N and M671L mutations, a V717F mutation, and a A692G mutation and a region of the human APP 3′-UTR.

The present invention provides for a recombinant nucleic acid molecule comprising a CaMKIIα promoter operatively linked to an APP gene comprising any of the mutations listed in Table 2 (all in accordance with the APP770 isoform). Table 2 lists the possible first, second and third mutations (i.e., amino acid substitutions) that can be present in the humanized mouse APP. It is readily appreciated by the skilled artisan that the designations, “first”, “second” and “third” in Table 2 are arbitrary and are not indicative of any specific order of the mutation, either in amino acid sequence or in the manner in which the constructs are made.

TABLE 2

| First Mutation | Second Mutation | Third Mutation |
|---|---|---|
| K670N/M671L | A692G | N/A |
| K670N/M671L | E693G | N/A |
| K670N/M671L | E693Q | N/A |
| K670N/M671L | A713T | N/A |
| K670N/M671L | I716V | N/A |
| K670N/M671L | V717I | N/A |
| K670N/M671L | V717F | N/A |
| K670N/M671L | V717G | N/A |
| K670N/M671L | A692G | E693G |
| K670N/M671L | A692G | E693Q |
| K670N/M671L | A692G | A713T |
| K670N/M671L | A692G | I716V |
| K670N/M671L | A692G | V717I |
| K670N/M671L | A692G | V717F |
| K670N/M671L | A692G | V717G |
| K670N/M671L | E693G | A713T |
| K670N/M671L | E693G | I716V |
| K670N/M671L | E693G | V717I |
| K670N/M671L | E693G | V717F |
| K670N/M671L | E693G | V717G |
| K670N/M671L | E693Q | A713T |
| K670N/M671L | E693Q | I716V |
| K670N/M671L | E693Q | V717I |
| K670N/M671L | E693Q | V717F |
| K670N/M671L | E693Q | V717G |
| K670N/M671L | A713T | I716V |
| K670N/M671L | A713T | V717I |
| K670N/M671L | A713T | V717F |
| K670N/M671L | A713T | V717G |
| K670N/M671L | I716V | V717I |
| K670N/M671L | I716V | V717F |
| K670N/M671L | I716V | V717G |

N/A: Not Applicable

Further embodiments of the present invention are humanized mouse APP polypeptides produced from the recombinant nucleic acid molecules described herein.

One embodiment of the present invention is a cell line which has been stably transformed by the recombinant nucleic acid molecules described herein. The cell line may be a human, mouse or rat cell line. The cell line may be a human cell line or a human neuronal cell line.

The present invention also provides for a transgenic nonhuman mammal whose germ or somatic cells contain a nucleic acid molecule which encodes a recombinant nucleic acid molecule as described herein, introduced into the mammal, or an ancestor thereof, at an embryonic stage. The nucleic acid molecule which is the transgene of the transgenic nonhuman mammal may contain an appropriate piece of genomic clone DNA from the mammal designed for homologous recombination.

The methods used for generating transgenic mice are well known to one of skill in the art. For example, methods are included in the manual entitled “Manipulating the Mouse Embryo” by Brigid Hogan et al. (Ed. Cold Spring Harbor Laboratory) (1986). The genetic engineering of non-human mammals (or any other organism) may be carried out according to at least two fundamentally different approaches: (1) random insertion of an exogenous gene into a host organism, and (2) gene targeting.

Transgenic non-human mammals resulting from the gene targeting technique, wherein a selected native DNA sequence or gene (i.e., targeted gene) is partially or completely removed or replaced through a process known as homologous recombination are also known. For a general description of gene targeting, see, e.g., Nature 336:348 (1988). One advantage of this technique is that if the targeted gene is a single-copy gene and the organism is homozygous at that locus, the gene-targeted organism can no longer express the targeted native gene, which can sometimes interfere and/or complicate transgenic studies.

Another embodiment of the present invention is a recombinant nucleic acid molecule, as described herein, comprising homologous regions to the native gene sequence to be used in the gene targeting technique to generate transgenic nonhuman mammals.

Another embodiment of the present invention is a method of evaluating whether a compound is effective in treating symptoms of a neurological disorder in a subject which comprises: (a) administering the compound to a transgenic nonhuman mammal of the invention, and (b) comparing the neurological function the mammal in step (a) with neurological function of the transgenic mammal in the absence of the compound, thereby determining whether the compound is effective in treating symptoms of the neurological disorder in a subject. In a further embodiment, the neurological function of the animal is assessed by the animal's performance in a memory or learning tests.

The neurological disorder may be amnesia, Alzheimer's disease, amyotrophic lateral sclerosis, a brain injury, cerebral senility, chronic peripheral neuropathy, a cognitive disability, a degenerative disorder associated with learning, Down's Syndrome, dyslexia, electric shock induced amnesia or amnesia, Guillain-Barre syndrome, head trauma, Huntington's disease, a learning disability, a memory deficiency, memory loss, a mental illness, mental retardation, memory or cognitive dysfunction, multi-infarct dementia and senile dementia, myasthenia gravis, a neuromuscular disorder, Parkinson's disease, Pick's disease, a reduction in spatial memory retention, senility, or Turret's syndrome.

The present invention provides for a method of evaluating whether a compound is effective in treating symptoms of a neurological disorder in a subject which comprises: (a) contacting a mammalian cell of the invention with the compound, and (b) comparing the neuronal cell function of the neuronal cell in step (a) with neuronal cell function in the absence of the compound, thereby determining whether the compound is effective in treating symptoms of the neurological disorder.

The nonhuman mammals of this invention may be used as tools or models to elucidate the role of human Aβ in AD pathology and symptomatology. The nonhuman mammals of this invention also may be used as assay systems to screen for in vivo inhibitors of amyloidogenic processing of APP to yield the human Aβ peptide in their brains, non-brain tissues, or body fluids (e.g., blood and cerebrospinal fluid).

The examples herein describe the actual construction of a recombinant nucleic acid molecule comprising a CaMKIIα promoter operatively linked to a humanized mouse APP gene comprising at least one mutation and a region of the human APP 3′-UTR. One of ordinary skill in the art will recognize that numerous other nucleic acid molecules could be designed to introduce the different mutations.

One of ordinary skill in the art will also recognize that various methods for producing murine, and non-murine, non-human mammals are known, and other strategies will be readily apparent. Furthermore, as new methods become available, additional strategies and targeting vectors will be apparent, and may be preferred. Accordingly, the following examples are not intended as, and are not to be construed as, limiting with respect to the disclosure or the scope of the claims. Other non-murine, nonhuman mammals are within the scope of the present invention.

It should be recognized from the foregoing discussion that the practice of the present invention requires a DNA clone comprising at least that region of the APP gene that includes the nucleotides to be replaced. Such necessary DNA clones may be obtained by a variety of means. The nucleotide sequence of the human APP gene is known. See, e.g., Kang et al. (supra); Goldgaber et al. (supra); Tanzi et al. (supra); and Robakis et al. (supra). The necessary DNA clones may be obtained, for example, by following the APP gene cloning methods set forth in the publications cited above. Alternatively, the published sequences can be used for the complete chemical synthesis of the desired DNA or the chemical synthesis of oligonucleotides that can be used as probes or PCR primers, as tools to obtain the necessary DNA by conventional techniques.

The compound may be an organic compound, a nucleic acid, a small molecule, an inorganic compound, a lipid, a peptide or a synthetic compound. The mammal may be a mouse, a goat, a sheep, a bovine, a canine, a porcine, or a primate. The subject may be a human. The administration may comprise intralesional, intraperitoneal, intramuscular or intravenous injection; infusion; liposome-mediated delivery; gene bombardment; topical, nasal, oral, anal, or ocular delivery.

In order that the invention described herein may be more fully understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, Cold Spring Harbor Laboratory (1982) or Sambrook et al., Molecular Cloning—A Laboratory Manual, 2nd Ed., Cold Spring Harbor Press (1989), using commercially available enzymes, except where otherwise noted.

### EXAMPLES

### Example 1

### Cloning Mutagenizing and Humanizing Mouse APP 695 Isoform

Mouse APP 695 isoform was cloned by PCR Mouse Brain Quick-Clone cDNA (Clontech) with the following primers:

| | |
|---|---|
| mmAPP-1: | |
| 5′ ATCTTCCACT CGCACACGGA GCACTCGGTG | (SEQ ID NO. 1) |
| 3′ | |
| mmAPP-2: | |
| 5′ GCGGGTGGGG CTTAGTTCTG CATTTGCTCA | (SEQ ID NO. 2) |
| AAG 3′ | |

The resulting ˜2.1 kb fragment was purified and cloned into pcDNA3.1 V5/His TOPO vector (Invitrogen) and sequencing confirmed it to be the APP 695 isoform.

The Quick-Change kit (Stratagene) was used to mutagenize and humanize the mouse APP 695 isoform. To introduce Indiana mutation, V642F (695 isoform), the following primers were used to PCR the plasmid containing wild type mouse APP 695 isoform:

| | |
|---|---|
| In forward: | |
| 5′ GCAACCGTGATTTTCATCATCACCCTGG 3′ | (SEQ ID NO. 3) |
| In reverse: | |
| 5′ CCAGGGTGATGAAAATCACGGTTGC 3′ | (SEQ ID NO. 4) |

The mutation was then confirmed by sequencing and named mouse APP 695 (In).

To introduce the Swedish mutation, K595N and N596L, and change G601R (695 isoform), the following primers were used to PCR the plasmid containing mouse APP 695 (In):

| | |
|---|---|
| Sw/G to R forward: | |
| 5′ CTCGGAAGTG AACCTGGATG CAGAATTCAG | (SEQ ID NO. 5) |
| ACATGATTCA G 3′ | |
| Sw/G to R reverse: | |
| 5′ CTGAATCATG TCTGAATTCT GCATCCAGGT | (SEQ ID NO. 6) |
| TCACTTCCGA G 3′ | |

The mutation was then confirmed by sequencing and named mouse APP 695 (In+Sw).

To further humanize the mouse APP 695 (In+Sw) by introducing F606Y and R609H mutations (695 isoform), the following primers were used to PCR the plasmid containing mouse APP 695 (In+Sw):

| | |
|---|---|
| F to Y/R to H forward: | |
| 5′ GATTCAGGAT ATGAAGTCCA CCATCAAAAA | (SEQ ID NO. 7) |
| C 3′ | |
| F to Y/R to H reverse: | |
| 5′ GTTTTTGATG GTGGACTTCA TATCCTGAAT | (SEQ ID NO. 8) |
| C 3′ | |

The mutation was then confirmed by sequencing and named humanized mmAPP (In−Sw).

### Example 2

### Cloning the 3′ UTR of Human APP Gene

To further increase the expression of the humanized mmAPP (In−Sw) transgene, the human APP 3′UTR was connected to the transgene described in Example 1. To clone the human APP 3′UTR, the following primers were used to PCR human hippocampus Quick-Clone cDNA (Clontech):

| | |
|---|---|
| hAPP-1: | |
| 5′ GCTCCTCCAA GAATGTATTT ATTTAC 3′ | (SEQ ID NO. 9) |
| hAPP-2: | |
| 5′ GCCACAGCAG CCTCTGAAG 3′ | (SEQ ID NO. 10) |

The resulting ˜1.1 kb fragment was cloned, and sequencing confirmed as the 3′UTR of human APP (data not shown).

### Example 3

### Connecting Humanized mnAPP (In−Sw) Transgene with Human APP 3′ UTR

To connect humanized mmAPP (In−Sw) transgene with human APP 3′ UTR, a PCR approach was used. First, the following primers were used to PCR amplify the human APP 3′UTR:

| mmAPP3′UTR-1: | |
|---|---|
| 5′ GCAGAACTAA GCCCCACCCG CAGCAGCCTC | (SEQ ID NO. 11) |
| TGAAGTTGGA CTGTAAAAC 3′ | |
| mmAPP3′UTR-4: | |
| 5′ GCTCCTCCAA GAATGTATTT ATTTACATG | (SEQ ID NO. 12) |
| 3′ | |

The resulting fragment was purified.

Second, the following primers were used to PCR amplify the humanized mmAPP (In−Sw) transgene:

| | |
|---|---|
| mmAPP3′UTR-3: | |
| 5′ CCACTCGCAC ACGGAGTACT C 3′ | (SEQ ID NO. 13) |
| mmAPP3′UTR-2: | |
| 5′ GTTTTACAGT CCAACTTCAG AGGCTGCTGC | (SEQ ID NO. 14) |
| GGGTGGGGCT TAGTTCTGC 3′ | |

The resulting fragment was also purified.

To connect humanized mmAPP (In−Sw) transgene and human APP 3′ UTR, the two PCR fragments containing human APP 3′UTR and humanized mmAPP (In−Sw) transgene, respectively were used as templates for PCR with primers mmAPP3′UTR-3 (SEQ ID NO. 13) and mmAPP3′UTR-4. (SEQ ID NO. 14) The resulting ˜3.3 kb fragment was cloned into pcDNA3.1 V5/His TOPO vector and confirmed via sequencing. The clone was named hmAPP(In−Sw)-hs3′UTR and the sequence is show in FIG. 3 and as SEQ ID NO. 15.

### Example 4

### Ligating of the hmAPP (In−Sw)-hs3′UTR Transgene to the CaMKIIα Promoter

The plasmid containing hmAPP(In−Sw)-hs3′UTR transgene was digested with EcoRV and BamHI. After digestion, Klenow was used to fill the BamHI sticky end. Then the fragment containing the transgene was gel purified. The vector plasmid, containing the CaMKIIα promoter, was linearized by NotI digestion and treated by Klenow. The transgene fragment was then ligated into the blunted NotI site after the CaMKIIα promoter by T4 DNA ligase. After overnight ligation, the resulting products were transformed into E. coli and plated on LB+AMP (100 ug/ml) plates. The positive clones were identified by colony hybridization.

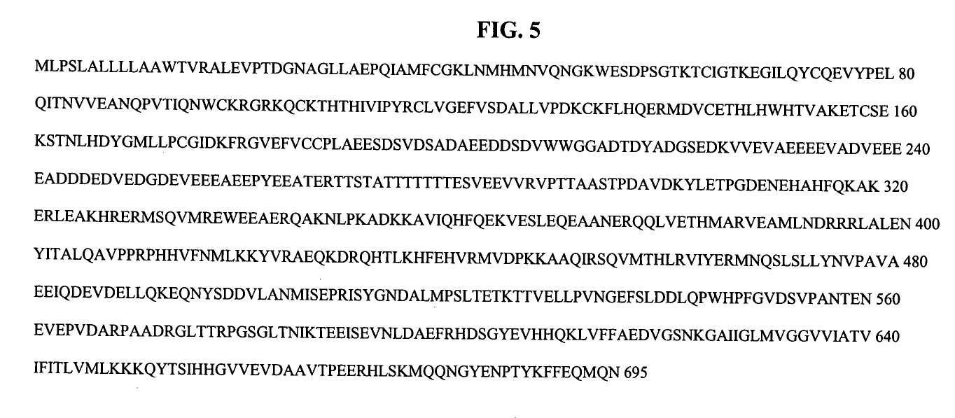

The plasmids from 22 positive colonies were recovered and digested with KpnI to check the orientation of the transgene. One plasmid with the right orientation (5′ end of the gene after the CaMKIIα promoter) was sequencing confirmed and named as pTG-ADi. The nucleic sequence of the CaMKIIα promoter-hmAPP(In−Sw)-hs3′UTR transgene is shown in FIG. 4 and as SEQ ID NO. 16. The CaMKIIα promoter sequence is located from nucleic acid number 1 to 8299 in FIG. 4. The hmAPP(In−Sw) gene sequence is located from nucleic acid number 8359 to 10450 in FIG. 4 and the hs3′UTR sequence is located from nucleic acid number 10458 to 11565 in FIG. 4. The amino acid sequence encoded from the hmAPP(In−Sw) gene sequence is indicated in FIG. 5 and SEQ ID NO. 17.

Other embodiments are within the following claims.

What is claimed is:

1. A recombinant nucleic acid molecule comprising a humanized mouse β-amyloid precursor protein (APP) gene comprising K670N, M671L and V717F mutations.

2. The recombinant nucleic acid molecule of claim 1, further comprising the calcium-calmodulin-dependent kinase IIα (CaMKIIα) promoter operatively linked to the humanized mouse APP gene.

3. The recombinant nucleic acid molecule of claim 1, further comprising a region of a 3′ untranslated region (3′ UTR).

4. The recombinant nucleic acid molecule of claim 3, wherein the 3′UTR is APP 3′ UTR.

5. The recombinant nucleic acid molecule of claim 4, wherein the APP 3′UTR is human APP 3′ UTR.

6. The recombinant nucleic acid molecule of claim 3, wherein the molecule has a sequence which confuses the sequence of FIG. 3.

7. The recombinant nucleic acid molecule of claim 2, further comprising a region of a 3′ untranslated region (3′ UTR).

8. The recombinant nucleic acid molecule of claim 7, wherein the 3′UTR is APP 3′ UTR.

9. The recombinant nucleic acid molecule of claim 8, wherein the APP 3′UTR is human APP 3′ UTR.

10. The recombinant nucleic acid molecule of claim 7, wherein the molecule ha as sequence which confuses the sequence of FIG. 4.

11. The recombinant nucleic acid molecule of claim 9, wherein the nucleic acid molecule has a sequence which comprises the nucleic acid sequence in ATCC Accession No. PTA-6646.

12. A humanized mouse APP polypeptide coded for by the recombinant nucleic acid of claim 1.

13. The polypeptide of claim 12, wherein the peptide has a sequence which confuses the sequence of FIG. 5.

14. A mammalian cell line which has been stably transformed with the recombinant nucleic acid molecule of claim 1.

15. A human cell line which has been stably transformed with the recombinant nucleic acid molecule of claim 1.

16. A human neuronal cell line which has been stably transformed with the recombinant nucleic acid molecule of claim 1.

17. A transgenic nonhuman mammal whose germ or somatic cells contain a nucleic acid molecule which encodes the recombinant nucleic acid molecule of claim 1 introduced into the mammal, or an ancestor thereof, at an embryonic stage.

18. A transgenic nonhuman mammal whose germ or somatic cells contain a nucleic acid molecule which encodes the recombinant nucleic acid molecule of claim 2 introduced into the mammal, or an ancestor thereof, at an embryonic stage.

19. A transgenic nonhuman mammal whose germ or somatic cells contain a nucleic acid molecule which encodes the recombinant nucleic acid molecule of claim 7 introduced into the mammal, or an ancestor thereof, at an embryonic stage.

20. A method of evaluating whether a compound is effective in treating symptoms of a neurological disorder in a subject which comprises:
(a) administering the compound to the transgenic nonhuman mammal of claim 17, and
(b) comparing the neurological function of the mammal in step (a) with the neurological function of the transgenic mammal in the absence of the compound, thereby determining whether the compound is effective in treating symptoms of the neurological disorder in a subject.

21. The method of claim 20, wherein the neurological function of the animal is assessed by the animal's performance in a memory or learning test.

22. The method of claim 20, wherein the neurological disorder is amnesia, Alzheimer's disease, amyotrophic lateral sclerosis, a brain injury, cerebral senility, chronic peripheral neuropathy, a cognitive disability, a degenerative disorder associated with learning, Down's Syndrome, dyslexia, electric shock induced amnesia or amnesia. Guillain-Barre syndrome, head trauma, Huntington's disease, a learning disability, a memory deficiency, memory loss, a mental illness, mental retardation, memory or cognitive dysfunction, multi-infarct dementia and senile dementia, myasthenia gravis, a neuromuscular disorder, Parkinson's disease, Pick's disease, a reduction in spatial memory retention, senility, or Turret's syndrome.

23. The method of claim 20, wherein the compound is an organic compound, a nucleic acid, a peptide, a small molecule, an inorganic compound, a lipid, or a synthetic compound.

24. The method of claim 20, wherein the mammal is a mouse, a sheep, a bovine, a canine, a porcine, a goat, or a primate.

25. The method of claim 20, wherein the subject is a human.

26. A method of evaluating whether a compound is effective in treating symptoms of a neurological disorder in a subject which comprises:
(a) contacting a human neuronal cell of the mammalian neuronal cell line of claim 14 with the compound; and
(b) comparing the neuronal cell function of the neuronal cell in step (a) with neuronal cell function in the absence of the compound, thereby determining whether the compound is effective in treating symptoms of the neurological disorder.

27. A recombinant nucleic acid molecule comprising a CaMKIIα promoter operatively linked to an APP gene comprising at least one mutation.

28. The recombinant nucleic acid molecule of claim 27, wherein the mutation comprises K670N and M671L.

29. The recombinant nucleic acid molecule of claim 27, wherein the mutation comprises K670N, M671 L and V717F.

30. The recombinant nucleic acid molecule of claim 27, wherein the APP gene is a humanized mouse APP gene.

31. The recombinant nucleic acid molecule of claim 27, further comprising a region of a 3′ untranslated region (3′ UTR).

32. The recombinant nucleic acid molecule of claim 31, wherein the 3 ′UTR is APP 3′ UTR.

33. The recombinant nucleic acid molecule of claim 32, wherein the APP 3′UTR is human APP 3′ UTR.

34. A humanized mouse APP polypeptide coded for by the recombinant nucleic acid of claim 30.

35. A mammalian cell line which has been stably transformed with the recombinant nucleic acid molecule of claim 27.

36. A human cell line which has been stably transformed with the recombinant nucleic acid molecule of claim 27.

37. A human neuronal cell line which has been stably transformed with the recombinant nucleic acid molecule of claim 27.

38. A transgenic nonhuman mammal whose germ or somatic cells contain a nucleic acid molecule which encodes the recombinant nucleic acid molecule of claim 27 introduced into the mammal, or an ancestor thereof, at an embryonic stage.

39. A method of evaluating whether a compound is effective in treating symptoms of a neurological disorder in a subject which comprises:
(a) administering the compound to the transgenic nonhuman mammal of claim 38, and
(b) comparing the neurological function of the mammal in step (a) with the neurological function of the transgenic mammal in the absence of the compound, thereby determining whether the compound is effective in treating symptoms of the neurological disorder in a subject.

40. The method of claim 39, wherein the neurological function of the animal is assessed by the animal's performance in a memory or learning test.

41. The method of claim 39, wherein the neurological disorder is amnesia, Alzheimer's disease, amyotrophic lateral sclerosis, a brain injury, cerebral senility, chronic peripheral neuropathy, a cognitive disability, a degenerative disorder associated with learning, Down's Syndrome, dyslexia, electric shock induced amnesia or amnesia. Guillain-Barre syndrome, head trauma, Huntington's disease, a learning disability, a memory deficiency, memory loss, a mental illness, mental retardation, memory or cognitive dysfunction, multi-infarct dementia and senile dementia, myasthenia gravis, a neuromuscular disorder, Parkinson's disease, Pick's disease, a reduction in spatial memory retention, senility, or Turret's syndrome.

42. The method of claim 39, wherein the compound is an organic compound, a nucleic acid, a peptide, a small molecule, an inorganic compound, a lipid, or a synthetic compound.

43. The method of claim 39, wherein the mammal is a mouse, a sheep, a bovine, a canine, a porcine, a goat, or a primate.

44. The method of claim 39, wherein the subject is a human.

45. A method of evaluating whether a compound is effective in treating symptoms of a neurological disorder in a subject which comprises:
(a) contacting a human neuronal cell of the mammalian neuronal cell line of claim 35 with the compound; and
(b) comparing the neuronal cell function of the neuronal cell in step (a) with neuronal cell function in the absence of the compound, thereby determining whether the compound is effective in treating symptoms of the neurological disorder.


SEQUENCE LISTING (17 sequences)

SEQ ID NO: 1 - DNA, 30 bp, Mammalian
atcttccactcgcacacggagcactcggtg

SEQ ID NO: 2 - DNA, 33 bp, Mammalian
gcgggtggggcttagttctgcatttgctcaaag

SEQ ID NO: 3 - DNA, 28 bp, Mammalian
gcaaccgtgattttcatcatcaccctgg

SEQ ID NO: 4 - DNA, 25 bp, Mammalian
ccagggtgatgaaaatcacggttgc

SEQ ID NO: 5 - DNA, 40 bp, Mammalian
ctcggaagtgaacctggatgcagaattcagacatgattca

SEQ ID NO: 6 - DNA, 41 bp, Mammalian
ctgaatcatgtctgaattctgcatccaggttcacttccgag

SEQ ID NO: 7 - DNA, 31 bp, Mammalian
gattcaggatatgaagtccaccatcaaaaac

SEQ ID NO: 8 - DNA, 31 bp, Mammalian
gtttttgatggtggacttcatatcctgaatc

SEQ ID NO: 9 - DNA, 26 bp, Mammalian
gctcctccaagaatgtatttatttac

SEQ ID NO: 10 - DNA, 19 bp, Mammalian
gccacagcagcctctgaag

SEQ ID NO: 11 - DNA, 49 bp, Mammalian
gcagaactaagccccacccgcagcagcctctgaagttggactgtaaaac

SEQ ID NO: 12 - DNA, 29 bp, Mammalian
gctcctccaagaatgtatttatttacatg

SEQ ID NO: 13 - DNA, 21 bp, Mammalian
ccactcgcacacggagtactc

SEQ ID NO: 14 - DNA, 49 bp, Mammalian
gttttacagtccaacttcagaggctgctgcgggtggggcttagttctgc

SEQ ID NO: 15 - DNA, 3303 bp, Mammalian
ccactcgcacacggagcactcggtggcccacgcaggatcacgatgctgcccagcttggcactgctcctgctggccgcctggacggttcgggctctggaggtacccactgatggcaacgccgggctgctggcagaaccccagatcgccatgttctgtggtaaactcaacatgcacatgaatgtgcagaatggaaagtgggagtcagacccgtcagggaccaaaacctgcattggcaccaaggagggcatcttgcagtactgccaagaggtctaccctgaactgcagatcacaaacgtggtggaagccaaccagccagtgaccatccagaactggtgcaagcggggccgcaagcagtgcaagacacacacccacatcgtgattccttaccgttgcctagttggtgagtttgtgagcgacgcccttctcgtgcccgacaagtgcaagttcctacaccaggagcggatggatgtttgtgagacccatcttcactggcacaccgtcgccaaagagacatgcagcgagaagagcactaacttgcatgactatggcatgctgctgccctgcggcatcgacaagttccgaggggtagagtttgtatgctgcccgttggccgaggaaagcgacagcgtggattctgcggatgcagaggaggatgactctgatgtctggtggggtggagcggacacagactacgctgatggcagtgaagacaaagtagtagaagtcgccgaagaggaggaagtggctgatgttgaggaagaggaagctgatgatgatgaggatgtggaggatggggacgaggtggaggaggaggccgaggagccctacgaagaggccaccgagagaacaaccagcactgccaccaccaccacaaccaccactgagtccgtggaggaggtggtccgagttcccacgacagcagccagcacccccgacgccgtcgacaagtacctggagacacccggggacgagaacgagcatgcccatttccagaaagccaaagagaggctggaagccaagcaccgagagagaatgtcccaggtcatgagagaatgggaagaggcagagcgtcaagccaagaacttgcccaaagctgacaagaaggccgttatccagcatttccaggagaaagtggaatctctggaacaggaagcagccaatgagagacagcagcttgtagagacacacatggccagagttgaagccatgctcaatgaccgccgccgcctggccctcgagaattacatcactgcactgcaggcggtgcccccaaggcctcatcatgtgttcaacatgctgaagaagtacgtccgtgcggagcagaaagacagacagcacaccctaaagcattttgaacatgtgcgcatggtggaccccaagaaagctgctcagatccggtcccaggttatgacacacctccgtgtgatctacgagcgcatgaaccagtctctgtccctgctctacaatgtccctgcggtggctgaggagattcaagatgaagtcgatgagctgcttcagaaggagcagaactactccgacgatgtcttggccaacatgatcagtgagcccagaatcagctacggaaacgacgctctcatgccttcgctgacggaaaccaagaccaccgtggagctccttcccgtgaatggggaattcagcctggatgacctccagccgtggcacccttttggggtggactctgtgccagccaataccgaaaatgaagtcgagcctgttgacgcccgccccgctgctgaccgaggactgaccactcgaccaggttctgggctgacaaacatcaagacggaagagatctcggaagtgaacctggatgcagaattcagacatgattcaggatatgaagtccaccatcaaaaactggtgttctttgctgaagatgtgggttcgaacaaaggcgccatcatcggactcatggtgggcggcgttgtcatagcaaccgtgattttcatcaccctggtgatgttgaagaagaaacagtacacatccatccatcatggcgtggtggaggtcgacgccgccgtgaccccagaggagcgccatctctccaagatgcagcagaacggatatgagaatccaacttacaagttctttgagcaaatgcagaactaagccccacccgcagcagcctctgaagttggactgtaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaagggcaattccaccacactggactagtggatccgagctcggtaccaagctta

SEQ ID NO: 16 - DNA, 11668 bp, Mammalian
ccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctctaaggcccgggcggcctcgacggtatcgataagcttcgatcttttttccgtaaactcaataccaggctgatgtcccaccggatctgatggcttagggtggcagggaatctcagttcccctcagacactctccctttgctggttctcagggaggaggcaaggtcaagtcttcatctgtaggcacgtggagggagggcacagaagccctcagctgaatagggtgggacttggggaagggcagcaaccaggctgggttgcctgggtcacaatcctgcctctttcctgatgagtttcctttttgccctcaggttacctatagcagcattctgcctcaatctcacccctaagatgagctctggtgactttaggactccagtgtacacatgtgtctggggccatggcagggtttcttgctgaccttgtcaccttccagacaacttgagtccatgaccctctttccagctctctgtggtgctcttggatatcagctggagtatggccagctggctgctgctctgttgaacaactcaatgagagaacggacagggtaggctctgagaaatctttacgttcctggagcctcatgacttgggagcctagtggaattcttctcttttggtccccaacatctggggggagggggaactggctgagcctgagccactgtatagtgagggtgggggaaacagctgtgaaaggagcctttgatttggtcttgaacacagttcttccccacagggccttgatttccctacttgcaaaggagtagggaaatatgagccttggctctgcctacctcacgttgctggtgctgtagaaaactggccaggctgatggttgtggaggagcctgtgaacttgattaaagtgccattatccagaggcaagagatgctggtctgtgtgtgtgtgagtgtgtgtgtgtgtgtgtgtgtgtgtgtgtgtcttggagacctgtgtgagctcgatgctagcagaggggacagaaggtaggtgggaaggaatgaaggaatgaaggaaggaaagaaggaaggttgaaaggaagaaaggaaggaaggaaggaaggaaggaaggaaggaaggaaggaaggaaggaaggaaggaaggtcctgccacaggcttaccatgtagctgcaggcaaacccctgaccctctctgggcctaagtgtttctctacacacaatggatgattcaagagtccttacttttggtggttacaggcacccctgtgcacatttgcatctggggtgggggggacacaggcttggtagtgttgaggaggggggtggttgtagagcctgctagctgcacactgcgttctgcatatctcccttcaggtcccagtcggcgcagtgtgtgtaggcgaaggccctgctgtgaattttgaaaatagttatttttgtcactggcaaaggaggccctgttaggactcgtcagcttgtggatgagcgggatgggtggagtggggtgggtgcggtgccgccgtggggggttaccgctgcttgcagggttgcatcgcccaggcagtgactgaatcctgcatgagggcctggcctaggctgtggggaggagatgaccactgcgtcctagatctttccttagccctgtgcttcctttcttccttttttccttaagatttatttctaatgatgcgtaggttgtgtatttgtgtgtgggtatgtgcacttgaataaaggacccacagaggctatagacatcagatcctcctagagctggggttacagagggcgtgagttgtccaacatgggcaccggaaaataaacttatgtgctctacacgaccgagctgtctctccaacaccagccctcttcttttgacttttctcatctccctcacttgtatgttttcccttcctcgataatgctgatacccagatggtaggcacggcccaggataggcaggggtctctgcgctccagtggctgtagtggttttcctgcctgctctgaagaagacactggctaaggtggtgtcttagcctactgtatcctagaggtgggttattcatagtctgcccactgcccaacacactctaggcctgctggggcctattctaactctgcttgctggcttgccaccctggcacacagtgtaggcttccctgtagagccaggctttagagaactgtatgagtacttctctgagaactgctggaggggccctgcctgccaggacttttcaacttccagccctgtcatctatatcacttctgaggaccccgtgtgggggtcacgagaacaagaccatatgtagtgctttctgttctcccttgggtcccaggctctgaatcaatctggtcccaagatataagggatgattggtgctgaggctggtgtctgtttctgaagttttgaagacaagggttggctcaagcctccctgtgttcagtcctccactcaatgcagaactcagtgaactcagaattctcagcccagatgccagcatagcccagcatagcccagcatagcccaggagctactggagcatcagtttgaaaccaggtccgcaggaactagtgggcaacagtgtgtgaggccagtggtctttggggtattgtattgaattgagaggtcctgcttagcagtcagcatgcccacaacctgttctctacggtggtccggattcccctcagcaagcacacctgaatctttactacatcccagttcctggttggctcctgacttcgggttactatggctgtgatgaaacactacgactaaaagcaatgtggggaagaaagttaattttttcactcgaccttccatagacagggttcatcactaaaagcagagggcagaggaaaagatagctcagcggttaagagtgctgtctactcaacaaagaggtcttgagttcaattcccagcaaccacatggtggctcacaactgtctatcctgggatctgatgcccttttctggcatacaggtatacatacagatagaggactcatatacataaaataaataaataaatctttaaaagcaacaagggcagaaattcaagcagggcagggacccagaggccaaagctgacgcagaggccatggaggggtgttgcttactggcttgctcctcatggcttgctcagcctgttttcttatagaacccacaaccaccaggccagagatgggaccacccacaaagggcagagcctttccctatcaatcactaatgggaaaacatcctgcagtcggatcttatgaagacattttctcagctgagcttccctcctatcagataactctagcttgtgtcaaggtgacttaaaactagccagcacagcaccttatgctcacatcacctgggtccctttggagaggacatagttaaagggagcccagaggcagtccctaggccacaggtcttcattgcccctctctgggacggattagacaggctgcagacctgttagctggaagagttagattcaggcaagagcttgaatctttacctgatcctggctatggagtcctggcctctaatgatcagctccctaacaacccaatggagccatatacctgcctgggccacggctgtgtctcctcttctttcagacactcctggcttgcctaggacacaggctagcatcctgtcaatgccaggaaggggcacagcagggaaagagcaatgctgttggcctgactgccatcaactggtgtacctgttagagggcaacctctattctctgcaccttggttcctagctctaagggatatgtggcccctaaaggtcttcatagcttgatatgggaggcaggggggctaagaacagcgcaagagtggtgagcttgcacagacccggatttgatctctgggtgagtgaggaggaaatgagatgggggtgggggaagccctatttctagctgtcttagcataggaactgaacctccttctgcagggcctgtgtcactgcccctttcccccagggagggcccctgcacggggcacctcagggcacagccctttttccctccctcctctcttagacctggaattactcaacatcctgccctgactcagttgctctcccctcagaccctcacagtcttccttctcttctggcccacttttggctgagcctgcccccaactttttctgcccttagtgggacaggccccatggggaccattcagatggcacttttttccccccctggggtggttttctgtggtggtgccctattcaggcaactgcaagaccctgtggcatttagcatatgcatgagagcacatgaagaagctagctatccctgtgtgctgaggattgtaatcctctctcatccttcccttgtctcctggaacccagtccagcctcctgtccctcccgttgacacgagccaatgctggctcagcaaactccagggctcccacccctggccatcagcccttggcacacaggcttgtgcttgagtactgcacacgtgttgcagctggggtacacgtgctggactgttatgcctactgtggccccgggggtgtgtgggaagtctggcagaaccaatccctccatcccccgatgcaatcatcagcttattctctcacggccactcgggcatgcttgactccttgatgcccgccgccactaggcacagctgccagctttgtgggcacagaggatgtggcgaattagtggtcatgcctcctcagtggaatggcaattgcactcagcatgcaggtgtctaccaaaggcagtccctacatccccgatgtactctcgagacccatctaaggactagatctagtctctagaaggtcccatgcagatgtaagacagccctccacagggagattcttccagctagttctctattatcagatgggtctaagatcctaggacctgcctatcccttagccctgcattcagcgagagaaggggtaaagatgtgaggatgccagggaggaaggaaaagggcacaaggaagaaagaaagggaaggaagctggaagcatggaaggacaaagatggtgaccacagtagaattaggatcccatggttcctgtcagtggcttcctgtgccttcctgtgcctccctgagcccctggggcatcttctaaatgctttgctggcctctgagccaagcactgcataccatcccgtggggagtgacaggccagcactggtcaacgaggatgatggctacttttgttcacagggtaacatctccatggttacagcctttgcacattcctcttagtactttaccaatctcaaagcagttgccaagcccttgggccctaataagtgagggtcccagtgccctcttttttaaattccttgccatttgttttgcagaatttactgcaaataaagccaaccccaggcaatgtctaaaccatgagttaaccccccagcaaggtctcagagaactgtgccccagagagctgccaaggttcagggaggagtatgaggagacaggatttctagttccttaataattccttctgtctcagccactgtgttcatcttgtttcagccacaaaactacctttattggtaaggaacattatttacccagtttcacactttaagaggtccagagacgttaacacatcgattcaaaagcacagcctgtaagtcacatagccactgttagctgatcgacactatttcccctgggcaatggctgggtgattccagggatccccttgggaacaggctagagcactggctctcaacctgtgcgggtcgtgacctccttgaggggtaggggtgagggcagtgtcaaacaacccttttacaggagtcgtttaagaccgttgggaaaaaaaccagatatttgcattatttttcgtaacagaagcaagattatagttatggagtagtgacaaaaattatgttacagttggaggtcagcacagcatgaggaactgtatttaagggttgcggcattaggaaggttgagaatcactggcctagcggatctgaatcaggaacacggacgtacagctctgcgccactcctgccttcctctggtgcctctagccttgcccatggtgttctgggcctgcctgctacccaccagctgtgcggccctgtgagcacaggcctttctgctccgctctgaattgccacgttggcggcagaagcgggaagcgtattgtgcgcagaaacaaaacggagtggttttttttttcctttttctgaaggtggtaatggtgcaattagtggcgaagccatcaccccctcctccccggctcgcctccctccttcctctccacctcccttctctttctttcctgagaaaaaaagtggctgagttgaaaagatctcccgtcaatctttctgtaacggactcaggaagagggatagagggccccctaatgtttccagggtcctcgagcctcagttgggtcaggcacttgttggtgctggagaatattcaaaggtaccactatgttccccacaagggagttgagcaatggattctgaggagcaagtttgaaacagagaatttgcgttcccaggtcttgtgatctgccccttgttcactgggggacaaatgctggcatgagaccctgagacctctgctcagccacctttctctctctctctctctttctctctctctctctctctctctctctctctctctctctctctctctgtccttaatggaggtgtgtgtgtgttcaagaccaagctgcagtgttggagtgcttgtgggctcattttaaaacttccatgttttgccttctagaaactgaaacataagaaccccattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatagggacctggatactgacgaaggctcgcgaggctgtgagcagccacagtgccctgctcagaagccccaagctcgtcagtcaagccggttctccgtttgcactcaggagcacgggcaggcgagtggcccctagttctgggggcagcgggggatccactagttctagagcggccatctgcagaattgcccttccactcgcacacggagcactcggtggcccacgcaggatcacgatgctgcccagcttggcactgctcctgctggccgcctggacggttcgggctctggaggtacccactgatggcaacgccgggctgctggcagaaccccagatcgccatgttctgtggtaaactcaacatgcacatgaatgtgcagaatggaaagtgggagtcagacccgtcagggaccaaaacctgcattggcaccaaggagggcatcttgcagtactgccaagaggtctaccctgaactgcagatcacaaacgtggtggaagccaaccagccagtgaccatccagaactggtgcaagcggggccgcaagcagtgcaagacacacacccacatcgtgattccttaccgttgcctagttggtgagtttgtgagcgacgcccttctcgtgcccgacaagtgcaagttcctacaccaggagcggatggatgtttgtgagacccatcttcactggcacaccgtcgccaaagagacatgcagcgagaagagcactaacttgcatgactatggcatgctgctgccctgcggcatcgacaagttccgaggggtagagtttgtatgctgcccgttggccgaggaaagcgacagcgtggattctgcggatgcagaggaggatgactctgatgtctggtggggtggagcggacacagactacgctgatggcagtgaagacaaagtagtagaagtcgccgaagaggaggaagtggctgatgttgaggaagaggaagctgatgatgatgaggatgtggaggatggggacgaggtggaggaggaggccgaggagccctacgaagaggccaccgagagaacaaccagcactgccaccaccaccacaaccaccactgagtccgtggaggaggtggtccgagttcccacgacagcagccagcacccccgacgccgtcgacaagtacctggagacacccggggacgagaacgagcatgcccatttccagaaagccaaagagaggctggaagccaagcaccgagagagaatgtcccaggtcatgagagaatgggaagaggcagagcgtcaagccaagaacttgcccaaagctgacaagaaggccgttatccagcatttccaggagaaagtggaatctctggaacaggaagcagccaatgagagacagcagcttgtagagacacacatggccagagttgaagccatgctcaatgaccgccgccgcctggccctcgagaattacatcactgcactgcaggcggtgcccccaaggcctcatcatgtgttcaacatgctgaagaagtacgtccgtgcggagcagaaagacagacagcacaccctaaagcattttgaacatgtgcgcatggtggaccccaagaaagctgctcagatccggtcccaggttatgacacacctccgtgtgatctacgagcgcatgaaccagtctctgtccctgctctacaatgtccctgcggtggctgaggagattcaagatgaagtcgatgagctgcttcagaaggagcagaactactccgacgatgtcttggccaacatgatcagtgagcccagaatcagctacggaaacgacgctctcatgccttcgctgacggaaaccaagaccaccgtggagctccttcccgtgaatggggaattcagcctggatgacctccagccgtggcacccttttggggtggactctgtgccagccaataccgaaaatgaagtcgagcctgttgacgcccgccccgctgctgaccgaggactgaccactcgaccaggttctgggctgacaaacatcaagacggaagagatctcggaagtgaacctggatgcagaattcagacatgattcaggatatgaagtccaccatcaaaaactggtgttctttgctgaagatgtgggttcgaacaaaggcgccatcatcggactcatggtgggcggcgttgtcatagcaaccgtgattttcatcaccctggtgatgttgaagaagaaacagtacacatccatccatcatggcgtggtggaggtcgacgccgccgtgaccccagaggagcgccatctctccaagatgcagcagaacggatatgagaatccaacttacaagttctttgagcaaatgcagaactaagccccacccgcagcagcctctgaagttggactgtaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaagggcaattccaccacactggactagtggatcggccgccacggtcgaggccgcccgggccttactcgagggggggcccggtacccaattcgccctatagtg

SEQ ID NO: 17 - protein, 695 aa, Mammalian
MLPSLALLLLAAWTVRALEVPTDGNAGLLAEPQIAMFCGKLNMHMNVQNGKWESDPSGTKTCIGTKEGILQYCQEVYPELQITNVVEANQPVTIQNWCKRGRKQCKTHTHIVIPYRCLVGEFVSDALLVPDKCKFLHQERMDVCETHLHWHTVAKETCSEKSTNLHDYGMLLPCGIDKFRGVEFVCCPLAEESDSVDSADAEEDDSDVWWGGADTDYADGSEDKVVEVAEEEEVADVEEEEADDDEDVEDGDEVEEEAEEPYEEATERTTSTATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPHHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHPFGVDSVPANTENEVEPVDARPAADRGLTTRPGSGLTNIKTEEISEVNLDAEFRHDSGYEVHHQKLVFFAEDVGSNKGAIIGLMVGGVVIATVIFITLVMLKKKQYTSIHHGVVEVDAAVTPEERHLSKMQQNGYENPTYKFFEQMQN